(19) United States
(12) Patent Application Publication
(10) Pub. No.: US 2009/0186068 A1
(43) Pub. Date: Jul. 23, 2009

(54) Atomic plasma deposited coatings for drug release

(75) Inventors: Tiffany E. Miller, Minneapolis, MN (US); Daniel M. Storey, Minneapolis, MN (US); Barbara S. Kitchell, Holmes Beach, FL (US)

Correspondence Address: CHAMELEON SCIENTIFIC CORPORATION;AKA IONIC FUSION CORPORATION, 13355 10TH AVENUE NORTH, SUITE 108, PLYMOUTH, MN, 55441, US

(73) Assignee: Chameleon Scientific Corporation

(21) Appl. No.: 12/150,298

(22) Filed: Apr. 25, 2008

Related U.S. Application Data
(60) Provisional application No. 61/011,551, filed on Jan. 18, 2008.

(51) Int. Cl. A61F 2/00

(52) U.S. Cl. 424/426

(57) ABSTRACT

Nanophase single or multiple layer time release coatings over drugs attached to metal surfaces are described. The coatings are deposited over a drug attached to a porous metal substrate using an atomic plasma deposition procedure. Porosity of the substrate and the number of APD deposited layers controls drug release when the attached drug is exposed to an aqueous medium.

This application claims benefit of provisional application Ser. No. 61/011,551 filed Jan. 18, 2008.

### BACKGROUND OF THE INTENTION

1. Field of the Invention

The invention concerns atomic plasma deposited nanoporous surfaces over biomolecules on various substrates to allow a time release of the biomolecule.

2. Description of Background Art

Elution of bioactive agents from implanted and indwelling medical devices has particular importance in the development of effective methods for administering therapeutics. Control of drug elution may be key to success in stents and other indwelling medical devices, which ideally should be able to remain in the body for long periods after implantation without restenosis.

Stents are small tubes placed in a blood vessel to maintain patency; i.e., to hold the vessel open so blood flow is not blocked. Coronary artery stents are typically metal, or a metal mesh framework, which over the years have been extensively used in heart patients. Unfortunately, bare metal stents are foreign to the body and may cause an immune response. The stent itself may induce rapid cell proliferation over its surface leading to scar tissue formation.

Drug eluting stents have shown marked improvements in preventing the blood clots associated with stent thrombosis or “target lesion revascularization”. Two models of drug eluting stents are currently used. The CYPHER stent (Cordis) releases rapamycin, which has both immunosuppressive and antiproliferative properties. It is sold under the name Sirolimus and is used primarily as an immunosuppressive drug to prevent organ transplant rejection. The drug is produced by Streptomyces hygroscopicus and has the effect of blocking certain stages in the cell cycle G - - - S transition. The CYPHER stent is fabricated from stainless steel and is coated with a polymer that acts as a time-release carrier for the drug rapamycin.

The TAXUS™ Stent (Boston Scientific, Boston, Mass.) releases paclitaxel, which, like rapamycin, is an antiproliferative drug used primarily in cancer therapies. Paclitaxel interacts with microtubles so that the cell cannot undergo mitosis. The TAXUS™ Stent also utilizes a polymer drug carrier coated over a stainless steel substrate.

The benefits of drug-eluting stents are well recognized. Widespread use of these stents has resulted in significantly reducing restenosis of coronary arteries, which in the past was prevalent after coronary artery bypass graft surgery particularly with the use of bare metal stents. Nevertheless, stents fabricated from new materials or in new configurations (e.g., open scaffolding), would be desirable as drug carriers or matrices possibly improve drug efficacy or be improved carriers for newly developed drugs. Magnesium alloy stents, for example, may have some advantage over stainless steel stents; however this material has so far been reported as only tested in animals

Stents for coronary arteries are typically composed of metal and many have been developed with various coatings on the stent surface. The coatings not only protect the body from exposure to the metal but are also designed to release various drugs intended to inhibit or at least delay reclosing of the blood vessel in which the stent was placed. Multi layer coatings can be used, with one or more layers containing a drug or therapeutic agent, although coating thickness may lead to sloughing or provide foci for restenosis from surface cracks or other imperfections. Drug eluting layers when used in coronary stents most frequently contain immunosuppressive compounds although anti-thrombogenic agents, anti-cancer agents and anti-stenosis drugs have also been used. Well-known and studied immunosuppressive drugs include ciclosporin A, rapamycin, daclizumab, demethomycin, and the like.

The drugs selected for use as drug-releasing coatings are often imbedded or associated with a polymer matrix, which is co-coated on the stent surface. Commonly used polymers for example, are polyester lactides, polyvinyl alcohol, cellulose. Patent application publication No 2005/0043788 describes a metal stent coated with a tripolide dispersed within a polymer matrix. U.S. Pat. No. 6,939,376 describes an intravascular stent having a drug releasing coating composed of an immunosuppressive agent in a poly-dl-lactide polymer with a micro thick polymer undercoating on the stent. Polylactide polymers have also been used to prepare macrocyclic triene immunosuppressive coatings over a polymer underlayer, as described in U.S. Pat. No. 6,939,376. Not all polymers are biocompatible and some simply will not effectively coat the metals commonly used for fabricating stents and other medical implants.

Stent design has also been investigated, including various shapes for improved coating adherence and drug delivery. Development of more flexible materials such as metal mesh has improved stent function and in vivo adaptability. U.S. Pat. No. 7,135,038 addresses stent structures that can be coated with varying thicknesses in different segments of the stent.

Despite the many improvements in stent design, materials and matrices for drug coatings, stents are subject to failure, due to development of inflammation at the implantation site or more commonly to restenosis of the artery. (Heublein, et al., 2003). Metals such as tantalum and cobalt alloy based stents are under investigation as bare metal stents, although current thinking is that drug eluting stents are preferable because they minimize re-blockage in artery linings to a greater extent than bare-metal stents, particularly when used for FDA approved situations; i.e., “on-label.

### SUMMARY OF THE INVENTION

The present invention is directed to thin, porous metal oxide surface films that serve as time variable release coatings. A controlled number of atomic layers of a metal oxide, illustrated with titanium oxide, can be deposited over a biomolecule such as a drug using an atomic plasma deposition (APD) process. Thickness of the APD film can be adjusted to control elution rate of the underlying drug attached or adhered to a substrate surface.

Controlled drug release APD films are particularly suitable for drug-eluting stents. In one aspect of the invention, atomic plasma deposited layers of a metal oxide can be applied over a drug attached or adhering to a stent surface. The deposition is on an atomic scale such that each deposition can be considered in effect as a monolayer. Logically, a greater number of deposited layers increasingly hinders elution of a surface-attached drug, thus allowing customization of time release.

The invention is illustrated with a model test drug on a cobalt chromium substrate surface. When not covered with any APD deposited layer of titania, the drug elutes almost immediately. However, by applying an APD surface, the drug elution from the substrate or matrix is significantly reduced.

The present invention utilizes relatively low temperature deposition conditions to prepare thin nanoporous for thin surface growth, in contrast to other vapor depositions which are conducted at much higher temperatures. Porous surfaces can be cyclically deposited in thin layers, best described as monolayers. These APD surfaces can include metal oxides, metals, or combinations of metals and/or metal oxides.

Regardless of the nanostructural features of APD deposited coatings, it is clear that APD deposited coatings over drug-coated substrates have a distinct effect on drug release. Bare metal substrates, on which drug is deposited, show relatively rapid elution. Alumina or titania APD top coats slow elution initially by at least several hours. The number of cycled layers, or monolayers, appears to have a controlling effect with 10 layers having little effect on normal elution, while an increasing number of layers, on the order of 100s, show a definite effect in slowing elution.

Additional control of drug elution can be obtained by attaching a drug to a nanoroughened surface before applying an elution-controlling APD porous top coat. Previous work has demonstrated that nanostructured substrate surfaces are formed when materials are deposited from high energy plasmas, where the deposited materials, e.g., titanium, are metals. Biomaterials, including drugs and proteins, can be efficiently deposited and relatively firmly attached to these surfaces using a procedure such as described in U.S. Pat. No. 7,250,195. The nanoplasma deposition (NPD) method can be applied to the implant surface to add nanoroughness for biomolecule loading. This surface is preferably less than 100 nm thick.

The invention in one broad aspect concerns a substrate overlaid with a biomolecule eluting surface constructed of two or three layers, which can be described as a biolayer and a porous top coating or, where there are three layers, a nanorough surface, a biolayer, and a porous top coating, respectively. The layers may be formed on any substrate material including metals, polymers or ceramics, and are ideal for use on materials commonly used for medical implants, which are typically stainless steel, titanium, chromium cobalt or any of a variety of ceramics or polymers.

Multiple biomolecule eluting surfaces can be utilized in order to achieve the desired elution profiles. Additionally, the biolayer need not be limited to a single type of compound or biomolecule, nor does the one or more compounds need to be bioactive. A molecular plasma deposition (MPD) procedure allows deposition of molecules individually or simultaneously if more than one molecular species is desired.

Coated drug surfaces are of particular interest in view of the wide range of therapeutic agents available to address adverse interactions encountered with medical implants. Currently popular drugs for use in arterial stents, for example, include anti-thrombotic and immunosuppressive agents. Other specialized implants may benefit from anti-microbial agents or antiflammatory drug coatings.

A particularly advantageous feature of the invention is the relatively thin biolayer underlying the barrier layer. Many stents are multicoated with a protective polymer layer (the barrier layer over the substrate) followed by one or more layers (the biolayer) of polymer-attached or emeshed drug. Such multilayers add thickness to the lumen of a coated stent, which may exacerbate sloughing and can contribute to manufacturing cost and quality control.

The top layer, of the disclosed multilayer coatings is an APD deposited film of a metal oxide such as titania or alumina. As a top surface, the APD deposited layers function to some extent as a protective layer, but mainly act as a time release control for the underlying bioactive molecules comprising the biolayer. A set number of depositions; i.e. monolayers, will control the amount of drug elution, exemplified with rapamycin, from near 100% elution within 2 hr for untreated surfaces to a much slower release over a period of 12 hours with 150 APD deposited titania layers.

Underlying surfaces of the invention; i.e., the substrates to which biomolecules are attached or in contact with, can have distinct functions and features. A nanorough substrate surface, if used, can be a thin NPD deposited material such as any of a number of metals, ranging from 1 to up to 100 nm thick, depending on desired substrate coverage and roughness.

A biolayer on the substrate, whether nanorough or smooth, can be deposited by MPD to obtain a select coverage or activity. Biolayers may be any of several molecular types, including metals, proteins and many organic molecules. The procedure is described and exemplified in U.S. Pat. No. 7,250,195, herein incorporated by reference. The biolayer may also be applied using ink-jet printing, spin coating, dip-coating and similar methods well-known in the art.

The top or final layer forms an APD porous surface, which can be deposited to a thickness appropriate for a desired elution rate of one or more biomolecules. The overall top layer is thin, less than 1 nanometer to several hundred nanometers thick depending on the elution rate desired. Overall thickness of the substrate coatings (the biomolecule and the top surface APD material) and the types of biomolecule(s) will determine the elution rate.

An advantage of selecting titania as a top APD layer is titania's recognized compatibility in vivo and its track record of use in medical implants. Titania is nontoxic and is not associated with an immune response.

The base substrate can be selected from a metal, ceramic or polymer, depending on use. For example, a biomolecule or other agent can be attached to or coated over gold, or silicon where applications as biosensors are contemplated.

Typical substrate materials used in medical devices such as orthopedic implants, dental implants, catheters and indwelling permanent or long-term devices include metals and plastics. Stainless steel, titanium and cobalt chromium stents are of particular interest in view of widespread use in heart vessel replacements. An additional advantage of APD titania top layers, as discussed, is that release of bioactive materials from substrate surfaces can be tailored to the properties of the underlying biomolecule.

While titania is exemplary of metal oxides that can be APD deposited, other metals are expected to exhibit similar properties, including alumina. Data are not shown for alumina, which appears to have some properties similar to titania. It is believed that other metals such as hafnium, iridium, platinum, gold, and silver can be produced as thin surface films with analogous properties.

### BRIEF DESCRIPTION OF THE FIGS.

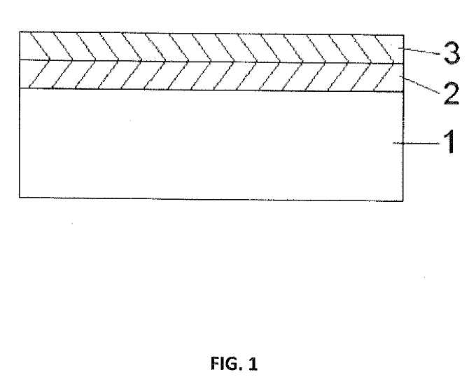

FIG. 1 illustrates the arrangement of a substrate (1) coated with a biomolecule (2) and overlaid with APD deposited titanium oxide (3) that allows elution of the biomolecule.

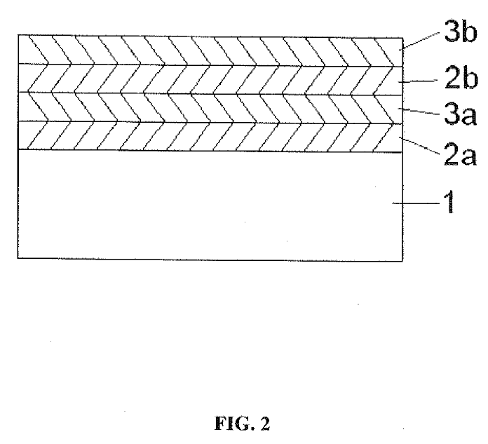

FIG. 2 illustrates a biomolecule-eluting system with repeating layers of biomolecule and coating on a substrate surface (1); layers of biomolecule (2a and 2b) overlaid with an APD titanium oxide film (3a and 3b) over each layer of drug.

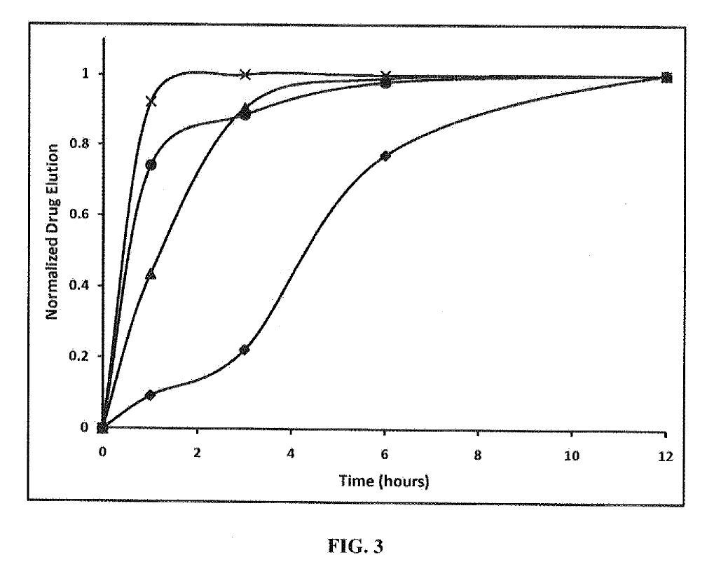

FIG. 3 is a graph showing an elution profile for rapamycin deposited on a substrate covered with APD titania, in the arrangement illustrated in FIG. 1. X represents the control without the APD titanium oxide coating film over the rapamyxin; , ▴, ▪ and represent APD deposited titania surface films of thicknesses 25 nm, 50 nm and 75 nm respectively.

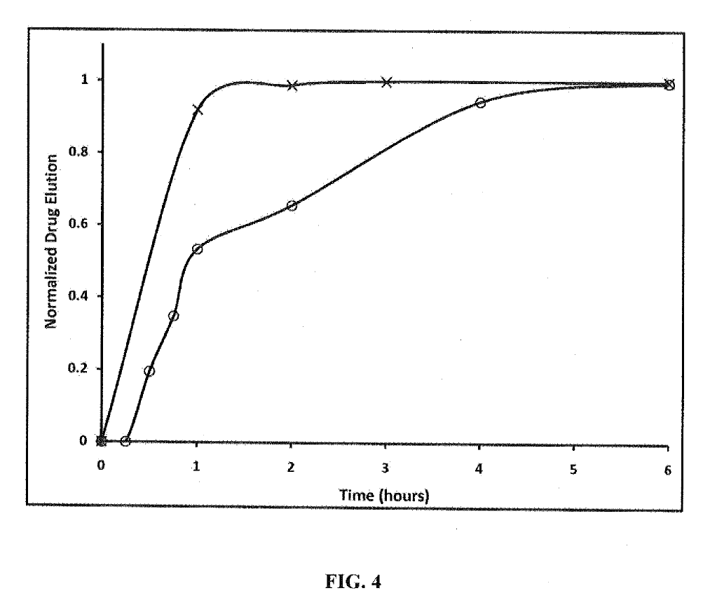

FIG. 4 is a graph showing a rapamycin elution profile from the multilayer system illustrated in FIG. 2. X is the control with only the drug applied to the substrate; O corresponds to elution of the drug applied in two layers, the first layer 2a covered with a 30 nm thick APD titanium oxide film 3a and the second layer 2b covered with a 35 nm thick APD titanium oxide film 3b.

### DETAILED DESCRIPTION OF THE INVENTION

Background of Atomic Plasma Deposition (APD)

The present invention utilizes an atomic plasma deposition (APD) technique that produces nanoscale thickness films on surfaces. The surfaces are produced using a modified plasma deposition technique to achieve surfaces ranging from sub-nanometer thicknesses up to hundreds of nanometers.

Ionic Plasma Deposition (IPD) is the vacuum deposition of ionized material generated in a plasma, generally by applying high voltage to a cathode target where the ionized plasma particles are deposited on a substrate which acts as an anode.

Nanoplasma deposition (NPD) utilizes an ionized gas produced by a DC current in order to deposit the ionized species onto a selected substrate surface. The thickness of films and coatings produced in this manner can be controlled but deposition is not uniform. This results in a nanorough surface.

MPD or molecular plasma deposition also utilizes a plasma, but produces the plasma from solutions or suspensions of materials introduced between the high voltage cathode and substrate anode, set up in a manner similar to IPD.

The invention provides methods for preparing nanoporous surfaces over immobilized or otherwise attached molecules on an underlying surface. The APD deposited metal oxide serves to protect the underlying biomolecule, but because such a thin film forms, will not prevent elution of the biomolecule. Of course elution rate is determined by more than the mere of a porous thin film. Factors, in addition to the thickness of the film and the metal oxide used, include the species of biomolecule, the nature and degree of biomolecule adherence to the underlying substrate, and the fluid environment to which the APD coated material is exposed. In most applications, it is desirable to use APD coatings over drugs well characterized as to activity and ability to attach to substrates recognized as appropriate for in vivo use.

The thickness of APD materials can be readily controlled by cycling the deposition conditions. For the exemplary drug rapamycin, described in the examples, relatively thin layers in the range of 25 to about 75 nm thickness provided a range of elution profiles, indicating that it is simply a matter of routine to determine appropriate thickness of the porous topcoat, in this case titania, but it could also be other metals such as aluminum oxide, for a desired elution rate. It should be noted that APD surface film thickness is not the sole factor to be considered in achieving a desired elution. Elution rates will necessarily depend on the chemical characteristics of the biomolecule and on the adhesion or binding of the biomolecule to the base substrate. The biomolecule can be covalently attached to the substrate, as would be possible on a gold substrate or on some metal substrates with activated surfaces. Substrates are not necessarily metal, and polymeric substrates could be combined with bioactive molecules. For most applications, biomolecules that are irreversibly bound to matrix and exhibit little or no elution are unlikely to benefit from nanoporous overcoats.

As mentioned, the biomolecule adhesion to the matrix or substrate is a factor in its elution characteristics. Several methods of contacting biomolecules to a surface are known. These include spraying, dipping, ink jet printing, and deposition methods such as molecular or nanoplasma deposition. Adherence or binding of the biomolecule may also be affected by the substrate material itself as well as surface roughness. Non-covalent interactions may be enhanced on nanorough surfaces. Surface area can be increased by mechanical means or by laser or plasma surface exposure. One method is to plasma deposit a metal onto a substrate so that the surface is pickled with micro or nanoparticulates. Adherence to these surfaces tends to be better than to smooth surfaces.

On the other hand, the disclosed APD titania nanoporous surfaces may well be appropriate as protective surfaces for mitigation of potential toxic effects from certain plastics or polymers that are in contact with the body. It is conceivable that a toxic material could be controllably eluted from an indwelling probe or other device in such a manner that the toxic agent is targeted, either by positioning of the device and/or because a targeting material is included; e.g., a targeting vector or antibody.

An additional advantage of the APD titania surfaces is their very thin profiles, which are resistant to sloughing. This is not only economical but at least in the case of titania, also provides a surface which consists of an inert material that is not known to be immunogenic and is not toxic.

### EXAMPLES

The following examples are provided as illustrations of the invention and are in no way to be considered limiting.

Materials.

Rapamycin was purchased from L.C. Laboratories (Woburn, Mass.) and used without further purification. Elution tests were performed in a 60% 1× phosphate buffered saline (PBS) and 40% methanol solution.

### Example 1

### Atomic Plasma Deposition of Thin Films

Metal oxide films can be deposited on various substrates by atomic plasma deposition (APD). In a typical example, titanium oxide was deposited in self limiting reactions from a reaction chamber supplied with alternating exposures of volatilized 30% hydrogen peroxide (in water) and titanium isopropoxide, using nitrogen as the carrier gas. To produce the titanium oxide, the following reaction sequence was used: 0.12 second exposure of hydrogen peroxide, 80 second delay, 0.12 second exposure of titanium isopropoxide, 80 second delay. The temperature of the reaction chamber was 50° C. Deposited film thickness depended on the number of cycles conducted.

### Example 2

### Metal Oxide Films on Biomolecule Coated Substrate

Using the APD method described in Example 1, titanium oxide thin films were grown over rapamycin which had been deposited on a stainless steel substrate. The rapamycin was deposited onto the substrate by the MPD method described in U.S. Pat. No. 7,250,195. The APD titania was grown over the rapamycin by sequential self-limiting reactions of titanium isopropoxide or trimethylaluminum and an oxygen source. FIG. 1 is a schematic illustration of the relative thicknesses of the rapamycin coated substrate and the overlying surface formed from the APD deposited titania.

FIG. 3 shows the amount of rapamycin elution from APD deposited titania of various thickness normalized to the control without the APD titania. , ▴, ▪ and represent APD deposited titania surface films of thicknesses 25 nm, 50 nm and 75 nm respectively with respective release of the drug over up to about 6 hr for the 25 and 50 nm thick layers and up to about 12 hr for 75 nm thick top layer. The rate of drug release into a PBS/methanol solution is roughly proportional to the thickness of the surface deposited material, at least for layers up to about 100 nm thick.

Example 3

### Biomolecule Release from Modified Substrate Surfaces

A titanium oxide film was deposited over rapamycin which had been applied to a cobalt chromium substrate. Rapamycin was deposited from a colloidal solution using the MPD procedure described. An APD coating of titanium oxide was deposited over the rapamycin using the APD process described in example 1 as depicted in the cross section of FIG. 1.

FIG. 4 is a rapamycin elution profile for release from a cobalt chromium substrate surface with APD deposited titania on two separate layers of MPD deposited rapamycin as depicted in the cross section of FIG. 2. In this example, the thicknesses of the titania layers 3a and 3b were 30 and 35 nm respectively. The control (x) has no top coating and the drug releases almost completely within about 2 hours. With the multiple layer coatings, rapamycin in initially released fairly rapidly, but then slows significantly compared with the control up to about 4.5 hr.

### REFERENCES

U.S. Pat. No. 5,697,967 (Dinh, T. Q., et al., 1997)

U.S. Pat. No. 7,135,038 (Limon, 2006)

U.S. Pat. No. 6,939,376 (Shulze, et al., 2005)

U.S. Pub. No. 2005/0043788 (Luo, et al.)

U.S. Pat. No., 7,250,195 (Storey, et al., 2007)

Heublein, B., Rhode, R., Kaese, V., Niemeyer, N., Hartung, W., and Haverich, “Biocorrosion of magnesium alloys: a new principle in cardiocasculr implant technology”, Heart 89: 651-656 (2003).

What is claimed is:

1. An atomic plasma deposited (APD) metal oxide surface layer that regulates elution rate of a biomolecule attached to or deposited on an underlying substrate.

2. The surface layer of claim 1 wherein the substrate is a metal, polymer, silicon or ceramic.

3. The surface layer of claim 1 which is about 1 to about several hundred nm thick.

4. The surface layer of claim 1 wherein the biomolecule is attached to the underlying substrate by dipping, ink jet, spray or plasma deposition.

5. The surface layer of claim 1 wherein the biomolecule is an antiproliferative drug.

6. The metal oxide surface layer of claim 1 wherein the metal oxide is titania.

7. A substrate comprising a deposited biomolecule over which is coated atomic plasma deposited (APD) titanium oxide.

8. The substrate of claim 7 which is selected from the group consisting of stainless steel, titanium, titanium alloy, magnesium alloy and cobalt alloy.

9. The substrate of claim 7 wherein the biomolecule is deposited by molecular plasma deposition, jet printing, spray, dipping or surface flooding.

10. A method for preparing a controlled release surface over a drug-attached substrate, comprising,
attaching a biomolecule to a substrate surface;
depositing by atomic layer deposition (APD) a titanium or alumina oxide surface having a thickness that releases the biomolecule over a selected period of time.

11. The method of claim 10 wherein the substrate comprises a nanoroughened surface.

12. The method of claim 10 wherein the biomolecule is deposited on the substrate surface by molecular plasma deposition from a solution or colloidal suspension.

13. The method of claim 12 wherein the biomolecule is an immunostimulatory or antiproliferative drug.

14. The method of claim 10 wherein the metal oxide is titania.

15. An atomic plasma deposited (APD) titanium or aluminum oxide surface coating over a biomolecule deposited or attached to an underlying substrate.

16. The APD coating of claim 15 wherein the biomolecule is deposited onto the substrate by molecular plasma deposition.

17. The APD coating of claim 15 wherein the biomolecule is an antiproliferative or immunosuppressive drug.

18. The APD metal oxide surface coating of claim 15 which is titania.

19. The APD coating of claim 18 which is between about 1 to about several hundred nm thick.

20. The APD coating of claim 18 which comprises titania from 1 up to about 500 nm thickness.

21. The APD coating of claim 18 which comprises titania from 1 up to about 100 nm thickness.
